(19) United States
(12) Patent Application Publication
(10) Pub. No.: US 2006/0271060 A1
(43) Pub. Date: Nov. 30, 2006

(54) Threaded knotless suture anchoring device and method

(75) Inventor: Norman S. Gordon, Irvine, CA (US)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/140,237

(22) Filed: May 26, 2005

(51) Int. Cl. A61B 17/58

(52) U.S. Cl. 606/103

(57) ABSTRACT

A bone anchor and methods for securing soft tissue, such as tendons, to bone, permits a suture attachment that lies entirely beneath the cortical bone surface. The suturing material between the soft tissue and the bone anchor is secured without the need for tying a knot, thus avoiding what is, for arthroscopic procedures, an extremely demanding and difficult task. A knotless anchor for fixation of soft tissues to bone includes a bone lock in the form of a screw, and a suture lock in the form of a plug which is movable into a lumen.

### BACKGROUND OF THE INVENTION

This invention relates generally to methods and apparatus for attaching soft tissue to bone, and more particularly to anchors and methods for securing connective tissue, such as ligaments or tendons, to bone. The invention has particular application to arthroscopic surgical techniques for reattaching the rotator cuff to the humeral head, in order to repair the rotator cuff.

It is an increasingly common problem for tendons and other soft, connective tissues to tear or to detach from associated bone. One such type of tear or detachment is a “rotator cuff” tear, wherein the supraspinatus tendon separates from the humerus, causing pain and loss of ability to elevate and externally rotate the arm. Complete separation can occur if the shoulder is subjected to gross trauma, but typically, the tear begins as a small lesion, especially in older patients.

To repair a torn rotator cuff, the typical course today is to do so surgically, through a large incision. This approach is presently taken in almost 99% of rotator cuff repair cases. There are two types of open surgical approaches for repair of the rotator cuff, one known as the “classic open” and the other as the “mini-open”. The classic open approach requires a large incision and complete detachment of the deltoid muscle from the acromion to facilitate exposure. The cuff is debrided to ensure suture attachment to viable tissue and to create a reasonable edge approximation. In addition, the humeral head is abraded or notched at the proposed soft tissue to bone reattachment point, as healing is enhanced on a raw bone surface. A series of small diameter holes, referred to as “transosseous tunnels”, are “punched” through the bone laterally from the abraded or notched surface to a point on the outside surface of the greater tuberosity, commonly a distance of 2 to 3 cm. Finally, the cuff is sutured and secured to the bone by pulling the suture ends through the transosseous tunnels and tying them together using the bone between two successive tunnels as a bridge, after which the deltoid muscle must be surgically reattached to the acromion. Because of this maneuver, the deltoid requires postoperative protection, thus retarding rehabilitation and possibly resulting in residual weakness. Complete rehabilitation takes approximately 9 to 12 months.

The mini-open technique, which represents the current growing trend and the majority of all surgical repair procedures, differs from the classic approach by gaining access through a smaller incision and splitting rather than detaching the deltoid. Additionally, this procedure is typically performed in conjunction with arthroscopic acromial decompression. Once the deltoid is split, it is retracted to expose the rotator cuff tear. As before, the cuff is debrided, the humeral head is abraded, and the so-called “transosseous tunnels”, are “punched” through the bone or suture anchors are inserted. Following the suturing of the rotator cuff to the humeral head, the split deltoid is surgically repaired.

Although the above described surgical techniques are the current standard of care for rotator cuff repair, they are associated with a great deal of patient discomfort and a lengthy recovery time, ranging from at least four months to one year or more. It is the above described manipulation of the deltoid muscle together with the large skin incision that causes the majority of patient discomfort and an increased recovery time.

Less invasive arthroscopic techniques are beginning to be developed in an effort to address the shortcomings of open surgical repair. Working through small trocar portals that minimize disruption of the deltoid muscle, a few surgeons have been able to reattach the rotator cuff using various bone anchor and suture configurations. The rotator cuff is sutured intracorporeally and an anchor is driven into bone at a location appropriate for repair. Rather than thread the suture through transosseous tunnels which are difficult or impossible to create arthroscopically using current techniques, the repair is completed by tying the cuff down against bone using the anchor and suture. Early results of less invasive techniques are encouraging, with a substantial reduction in both patient recovery time and discomfort.

Unfortunately, the skill level required to facilitate an entirely arthroscopic repair of the rotator cuff is inordinately high. Intracorporeal suturing is clumsy and time consuming, and only the simplest stitch patterns can be utilized. Extracorporeal knot tying is somewhat less difficult, but the tightness of the knots is difficult to judge, and the tension cannot later be adjusted. Also, because of the use of bone anchors to provide a suture fixation point in the bone, the knots that secure the soft tissues to the anchor by necessity leave the knot bundle on top of the soft tissues. In the case of rotator cuff repair, this means that the knot bundle is left in the shoulder capsule where it is able to be felt by the patient postoperatively when the patient exercises the shoulder joint. So, knots tied arthroscopically are difficult to achieve, impossible to adjust, and are located in less than optimal areas of the shoulder. Suture tension is also impossible to measure and adjust once the knot has been fixed. Consequently, because of the technical difficulty of the procedure, presently less than 1% of all rotator cuff procedures are of the arthroscopic type, and are considered investigational in nature.

Another significant difficulty with current arthroscopic rotator cuff repair techniques are shortcomings related to currently available suture anchors. Suture eyelets in bone anchors available today, which like the eye of a needle are threaded with the thread or suture, are small in radius, and can cause the suture to fail at the eyelet when the anchor is placed under high tensile loads.

There are various bone anchor designs available for use by an orthopedic surgeon for attachment of soft tissues to bone. The basic commonality between the designs is that they create an attachment point in the bone for a suture that may then be passed through the soft tissues and tied, thereby immobilizing the soft tissue. This attachment point may be accomplished by different means. Screws are known for creating such attachments, but suffer from a number of disadvantages, including their tendency to loosen over time, requiring a second procedure to later remove them, and their requirement for a relatively flat attachment geometry.

Another approach is to utilize the difference in density in the cortical bone (the tough, dense outer layer of bone) and the cancellous bone (the less dense, airy and somewhat vascular interior of the bone). There is a clear demarcation between the cortical bone and cancellous bone, where the cortical bone presents a kind of hard shell over the less dense cancellous bone. The aspect ratio of the anchor is such that it typically has a longer axis and a shorter axis and usually is pre-threaded with a suture. These designs use a hole in the cortical bone through which an anchor is inserted. The hole is drilled such that the shorter axis of the anchor will fit through the diameter of the hole, with the longer axis of the anchor being parallel to the axis of the drilled hole. After deployment in to the cancellous bone, the anchor is rotated 90 degrees so that the long axis is aligned perpendicularly to the axis of the hole. The suture is pulled, and the anchor is seated up against the inside surface of the cortical layer of bone. Due to the mismatch in the dimensions of the long axis of the anchor and the hole diameter, the anchor cannot be retracted proximally from the hole, thus providing resistance to pull-out. These anchors still suffer from the aforementioned problem of eyelet design that stresses the sutures.

Still other prior art approaches have attempted to use a “pop rivet” approach. This type of design requires a hole in the cortical bone into which a split shaft is inserted. The split shaft is hollow, and has a tapered plug leading into its inner lumen. The tapered plug is extended out through the top of the shaft, and when the plug is retracted into the inner lumen, the tapered portion causes the split shaft to be flared outwardly, ostensibly locking the device into the bone.

Other methods of securing soft tissue to bone are known in the prior art, but are not presently considered to be feasible for shoulder repair procedures, because of the reluctance of physicians to leave anything but a suture in the capsule area of the shoulder. The reason for this is that staples, tacks, and the like could possibly fall out and cause injury during movement. As a result of this constraint, the attachment point often must be located at a less than ideal position. Also, the tacks or staples require a substantial hole in the soft tissue, and make it difficult for the surgeon to precisely locate the soft tissue relative to the bone.

As previously discussed, any of the anchor points for sutures mentioned above require that a length of suture be passed through an eyelet fashioned in the anchor and then looped through the soft tissues and tied down to complete the securement. Much skill is required, however, to both place the sutures in the soft tissues, and to tie knots while working through a trocar under endoscopic visualization.

There have been attempts to solve some of the problems that exist in current anchor designs. One such approach is disclosed in U.S. Pat. No. 5,324,308 to Pierce. In this patent, there is disclosed a suture anchor that incorporates a proximal and distal wedge component having inclined mating faces. The distal wedge component has two suture thread holes at its base through which a length of suture may be threaded. The assembly may be placed in a drilled hole in the bone, and when tension is placed on the suture, the distal wedge block is caused to ride up against the proximal wedge block, expanding the projected area within the drilled hole, and locking the anchor into the bone. This approach is a useful method for creating an anchor point for the suture, but does not in any way address the problem of tying knots in the suture to fix the soft tissue to the bone.

The problem of placing sutures in soft tissues and tying knots in an endoscopic environment is well known, and there have been attempts to address the problem and to simplify the process of suture fixation. One such approach is disclosed in U.S. Pat. No. 5,383,905 to Golds et al. The patent describes a device for securing a suture loop about bodily tissue that includes a bead member having a longitudinal bore and an anchor member adapted to be slidably inserted within the bore of the bead member. The anchor member includes at least two axial compressible sections which define a passageway to receive two end portions of a suture loop. The axial sections collapse radially inwardly upon insertion of the anchor member within the bore of the bead member to securely wedge the suture end portions received within the passageway.

Although the Golds et al. patent approach utilizes a wedge-shaped member to lock the sutures in place, the suture legs are passing through the bore of the bead only one time, in a proximal to distal direction, and are locked by the collapsing of the wedge, which creates an interference on the longitudinal bore of the anchor member. Also, no provision is made in this design for attachment of sutures to bone. The design is primarily suited for locking a suture loop, such as is used for ligation or approximation of soft tissues.

An approach that includes bone attachment is described in U.S. Pat. No. 5,584,835 to Greenfield. In this patent, a two part device for attaching soft tissue to bone is shown. A bone anchor portion is screwed into a hole in the bone, and is disposed to accept a plug that has been adapted to receive sutures. In one embodiment, the suture plug is configured so that when it is forced into its receptacle in the bone anchor portion, sutures that have been passed through an eyelet in the plug are trapped by friction between the wall of the anchor portion and the body of the plug portion.

Although there is some merit to this approach for eliminating the need for knots in the attachment of sutures to bone, a problem with being able to properly set the tension in the sutures exists. The user is required to pull on the sutures until appropriate tension is achieved, and then to set the plug portion into the bone anchor portion. This action increases the tension in the sutures, and may garrot the soft tissues or increase the tension in the sutures beyond the tensile strength of the material, breaking the sutures. In addition, the minimal surface area provided by this anchor design for pinching or locking the sutures in place will abrade or damage the suture such that the suture's ability to resist load will be greatly compromised.

A disclosure that incorporates bone attachment and eliminates knot tying is set forth in U.S. Pat. No. 5,702,397 to Goble et al. One embodiment, in particular, is shown in FIG. 23 of that patent and includes a bone anchor that has a threaded body with an inner cavity. The cavity is open to one end of the threaded body, and joins two lumens that run out to the other end of the threaded body. Within the cavity is disposed a gear, journaled on an axle. A length of suture is threaded through one lumen, around the gear, and out through the other lumen. A ball is disposed within the cavity to ride against a tapered race and ostensibly lock the suture in place. What is not clear from the patent disclosure is how the force D shown as the tension in the suture would lock the ball into the race. Although this embodiment purports to be a self-locking anchor adapted for use in blind holes for fixing sutures into bone, the construct shown is complicated, and does not appear to be adequate to reliably fixate the suture.

The use of screws for the creation of the attachment point in the bone is well known in the art. Two patents among many that illustrate the broad application of screw shaped constructs are U.S. Pat. No. 5,851,219 to Goble et al and U.S. Pat. No. 6,117,162 to Schmieding et al. These two patents focus on particular aspects of suture anchors such as self tapping threads, or the shape of the threads, or a particular shape or configuration of the driving means. A common feature of these two patents, and indeed the majority of patents in the art of bone screw anchors is the inclusion of an eyelet for accommodation of the suture.

A screw anchor that does not use an eyelet is disclosed in U.S. Pat. No. 5,571,139 to Jenkins, Jr. wherein a cannulated or hollow screw anchor is disclosed. This anchor uses a stepped internal channel that will accommodate lengths of suture, but is small enough not to allow a knot placed in the suture to migrate through the anchor. Although this anchor does not use an eyelet, it still requires the creation of knots in the suture to lock the tissues in place.

Another screw anchor patent that discloses a knotless approach is U.S. Pat. No. 6,159,235 to Kim. One of the unique features of this anchor is the ability to rotate the anchor body to insert the screw while keeping the suture from wrapping up around the anchor. However, in looking at the embodiment described, a couple of problems are clear. The suture clamping area due to the geometry required by the ring and journal construction is somewhat limited, and it is expected that construction would cause a stress riser in the suture such that the suture would consistently break at the ring at a relatively low tension. Also, because the screw needs to be driven further into the bone in order to lock the suture, tension on the cuff will be increased as the screw is tightened. Although it may be that the locking ring and journal may be massaged to accomplish reasonable knot pull strength, the issue of tension is unavoidable. It may be mitigated with training and experience, but the fact remains that excess tension would be a common failure mode.

What is needed, therefore, is a new approach for repairing the rotator cuff or fixing other soft tissues to bone, wherein suture tension can be adjusted and possibly measured, the suture anchor resides completely below the cortical bone surface, there is no requirement for the surgeon to tie a knot to attach the suture to the bone anchor, and wherein the procedure associated with the new approach is better for the patient, saves time, is uncomplicated to use, and easily taught to practitioners having skill in the art.

### SUMMARY OF THE INVENTION

The present invention solves the problems outlined above by providing innovative bone anchor and connective techniques which permit a suture attachment which lies entirely beneath the cortical bone surface. In the present state of the art, the sutures which are passed through the tissues to be attached to bone typically are threaded through a small eyelet incorporated into the head of the anchor and then secured by tying knots in the sutures. Endoscopic knot tying is an arduous and technically demanding task. Therefore, the present invention discloses devices and methods for securing sutures to a bone anchor without the requirement of knot tying.

In particular, the present invention includes further improvements to the novel suture locking mechanism disclosed in co-pending U.S. patent application Ser. No. 09/781,793, entitled Method & Apparatus for Attaching Connective Tissues to Bone Using a Knotless Suture Anchoring Device, filed on Feb. 12, 2001, and presently allowed. The referenced application is commonly assigned with the present application, and is expressly incorporated by reference in its entirety herein.

As previously discussed, knot tying in arthroscopic procedures is an extremely demanding and difficult task. Elimination of this step in the performance of, for example, an arthroscopic rotator cuff repair, while still showing the advantages of using suture for attachment to the cuff, streamlines and simplifies the procedure. Advantageously, therefore, a knotless anchor is disclosed for fixation of soft tissues to bone, including a bone lock in the form of a screw, as well as a suture tensioning mechanism and a suture locking mechanism.

Now, it is to be understood that the above described invention is particularly suited to locking sutures that have been passed through soft tissues and are to be anchored to bone. The creation of an anchor point within the bone utilizing a screw construct is within the scope of this invention, although many alternative methods of anchoring suture to bone are contemplated. For example, some currently preferred methods are discussed in U.S. Pat. No. 6,582,453, and in U.S. Pat. No. 6,547,800. The referenced patents are both commonly assigned with the present application, and are expressly incorporated by reference, each in their entirety, herein. Other prior art anchors, such as moly bolts, and pop rivets may be adapted for use with the present invention as well.

More particularly, there is disclosed a knotless suture anchor apparatus for anchoring a length of suture with respect to a body cavity. This apparatus comprises an anchor body having an anchoring structure for fixing the anchor body within a body cavity. The anchoring structure comprises a threaded surface which is rotatable to engage adjacent bone. A suture tensioning mechanism for accommodating and tensioning the length of suture is also provided. Additionally, a suture locking mechanism for locking the length of suture in place, once it has been tensioned to a desired level, forms a part of the disclosed apparatus.

Preferably, the threaded surface is disposed on a distal end of the anchoring structure, and the anchoring structure further comprises a shaft extending proximally from the threaded surface. The proximal end of the shaft is connected to a handle. The shaft preferably comprises an inner, tubular shaft, and the anchoring structure further comprises an outer shaft disposed about the inner shaft. The outer shaft is proximally removable from its position disposed about the inner shaft after the threaded surface is engaged in the adjacent bone.

It should further be noted that the suture tensioning mechanism is structurally integrated with the anchoring structure, and is deployed after the threaded surface is engaged in the adjacent bone and after portions of the anchoring structure have been withdrawn. A snare loop is preferably employed for snaring the length of suture and threading it through the suture tensioning mechanism. Moreover, the suture tensioning mechanism comprises a rotatable knob which is operably connected to a ratchet and pawl system. The suture locking mechanism comprises a locking lever for actuating a rotatable cable capture plate.

In another aspect of the invention, there is disclosed a knotless suture anchor apparatus for anchoring a length of suture with respect to a body cavity, which comprises an anchor body having a screw-type anchoring structure for fixing the anchor body within a body cavity. The screw-type anchoring structure comprises a threaded surface on a distal end thereof which is rotatable to engage adjacent bone, wherein the anchoring structure further comprises a shaft extending proximally from the threaded surface. A handle is connected to a proximal end of the shaft. The apparatus further comprises a suture tensioning mechanism for accommodating and tensioning the length of suture, and a suture locking mechanism for locking the length of suture in place once it has been tensioned to a desired level.

In still another aspect of the invention, there is disclosed a method of securing soft tissue with respect to a body cavity without knots, which comprises a step of passing a length of suture through soft tissue so that a loop of suture material is embedded in the soft tissue resulting in two free ends, and a second step of threading the two free ends of the length of suture through an anchor body. The method further comprises an additional step of engaging a distal end of the anchor body with adjacent bone to fix the anchor body in place within the body cavity, tensioning the length of suture to approximate the soft tissue to the bone as desired; and locking the length of suture in position after it has been tensioned as desired.

In a preferred approach, the threading step includes snaring the length of suture. Moreover, a portion of the anchor body is removed after the engaging step.

The invention, together with additional features and advantages thereof, may best be understood by reference to the following description taken in conjunction with the accompanying illustrative drawing.

### BRIEF DESCRIPTION OF THE DRAWINGS

FIG. 1A is a partial sectional view through the left shoulder of a human as seen from the front showing the use of a minimally invasive soft tissue to bone attachment system of the present invention;

FIG. 1B is an enlarged sectional view taken within the circle denoted 1B in FIG. 1A;

FIGS. 2A-2D are enlarged sectional views of the use of the soft tissue to bone attachment system of FIG. 1A to reattach a rotator cuff tendon;

FIG. 3 is a perspective view of one embodiment of a suture anchoring device constructed in accordance with the principles of the present invention;

FIG. 4 is a perspective view from the opposite side of the device shown in FIG. 3, with portions removed to illustrate a first step in the inventive method;

FIG. 5 is a perspective view similar to FIG. 4, illustrating a second step in the inventive method;

FIG. 6 is a perspective view similar to FIG. 5, illustrating a third step in the inventive method;

FIG. 7 is a cross-sectional view of the device shown in FIGS. 3-6, illustrating further details of its construction;

FIG. 8 is a partial perspective view of a distal section of the handle portion of the device shown in FIGS. 3-7, illustrating constructional details of the suture tensioning system;

FIG. 9 is a perspective view of the entire handle portion of the device shown in FIGS. 3-8, illustrating a first step for tensioning the suture;

FIG. 10 is a perspective view similar to FIG. 9, illustrating a second step for tensioning the suture;

FIG. 11 is a perspective view similar to FIG. 10, illustrating a third step for tensioning the suture;

FIG. 12 is a perspective view of the device shown in FIGS. 3-11, illustrating a step of completing the inventive procedure by removing the bone anchor applying and suture tensioning mechanism from the procedural site;

FIG. 13 is a perspective view of a second embodiment of a suture anchoring device constructed in accordance with the principles of the present invention;

FIG. 14 is a perspective view of the embodiment of FIG. 13, illustrating a first step in the inventive method;

FIG. 15 is a perspective view of the embodiment of FIGS. 13 and 14, after the bone anchoring device has been removed, illustrating suture management techniques in accordance with the invention;

FIG. 16 is a perspective view of the embodiment of FIGS. 13-15, after a suture tensioning apparatus has been introduced into the procedural site;

FIG. 17 is a top view of the suture tensioning mechanism of the embodiment of FIGS. 13-16;

FIG. 18 is a perspective view of the embodiment of FIGS. 13-17, illustrating a suture tensioning step of the invention;

FIG. 19 is a perspective view of the suture tensioning mechanism of FIG. 17;

FIG. 20 is a perspective view of the embodiment of FIGS. 13-19, illustrating a further suture tensioning step of the invention;

FIG. 21 is a perspective view similar to FIG. 20 of the embodiment of FIGS. 13-20, illustrating yet a further suture tensioning step of the invention;

FIG. 22 is a perspective view similar to FIG. 21 of the embodiment of FIGS. 13-20, illustrating a final suture tensioning step of the invention;

FIG. 23 is a perspective view of an implant adapted to be delivered by the mechanisms described above;

FIG. 24 is a perspective view of the implant of FIG. 23, from a different orientation; and

FIGS. 25A, 25B, and 25C are cross-sectional views, taken sequentially, of the implant shown in FIGS. 23 and 24, illustrating the functional aspects of the structure.

### DESCRIPTION OF THE PREFERRED EMBODIMENT

The present invention provides an improved knotless suture anchor apparatus for anchoring a length of suture with respect to a body cavity. In the exemplary embodiment described herein, the apparatus is used to anchor a length of suture to a bone structure, specifically the humeral bone of the human shoulder. The length of suture is desirably looped through soft tissue, such as a rotator cuff tendon, to approximate and fix the soft tissue with respect to the body cavity (e.g., bone structure). It should be understood, however, that the suture anchor apparatus may be utilized to secure a length of suture to body cavities other than in a bone structure, and may even be used to anchor the suture outside of a body cavity, merely to a predetermined location within the body. In this regard, the preferred apparatus includes an anchor body within which the length of suture may be anchored without knots. If the anchor body is to be implanted within the body cavity, a screw anchor is provided for securing the anchor body therein.

As mentioned, the present invention is particularly well-suited for repairing rotator cuff injuries by re-attaching the rotator cuff tendon to the outside of the humeral head. The invention permits minimally invasive surgeries on such injuries and greatly facilitates rapid and secure fixation of the rotator cuff tendon to the humeral head. It should be understood that the same principles described herein apply to the repair of other injuries in which soft tissue is to be re-attached to a bone structure.

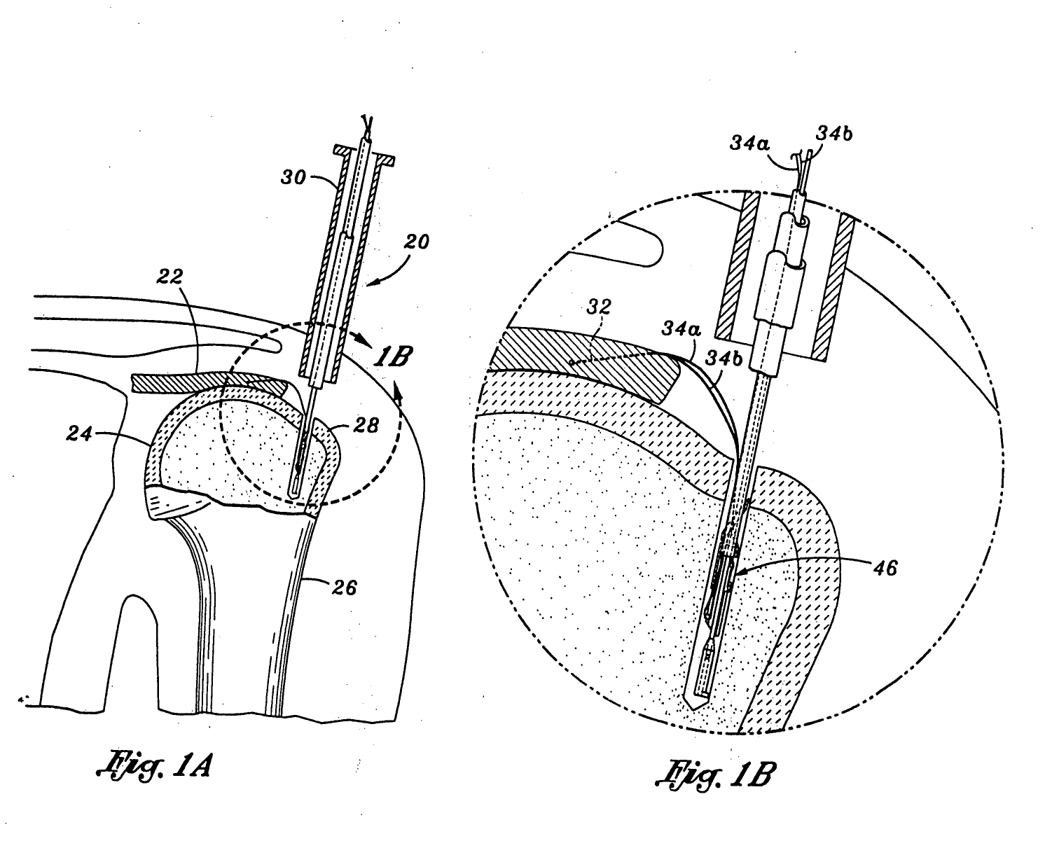

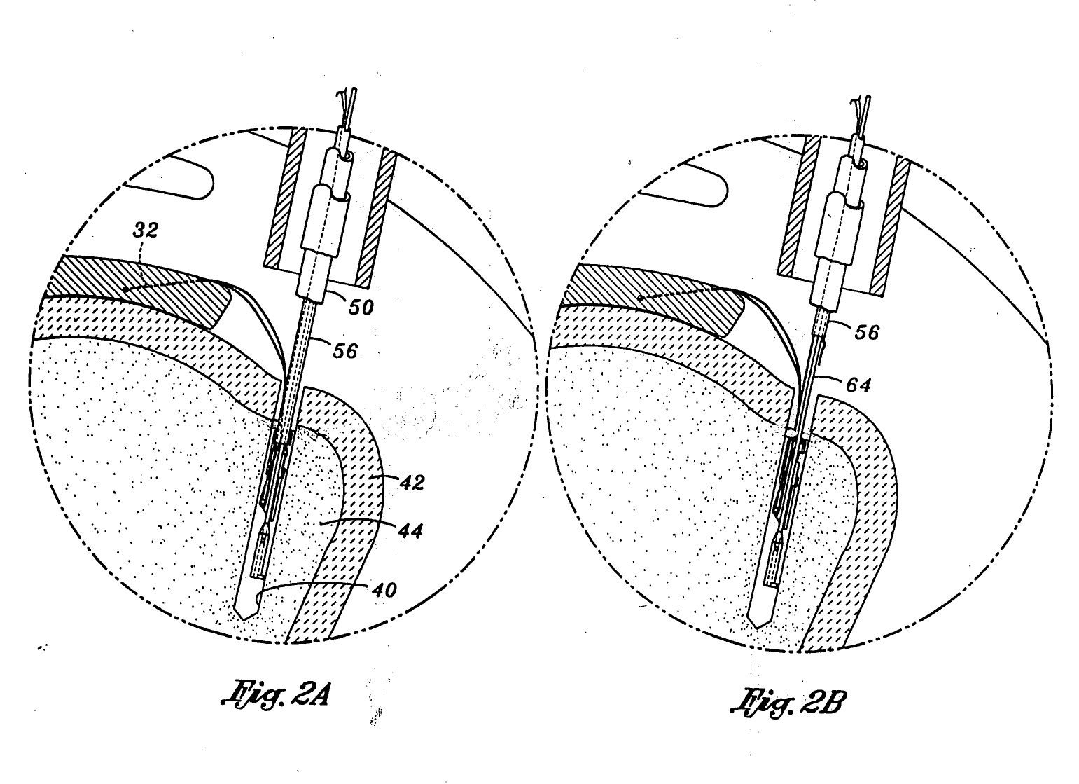

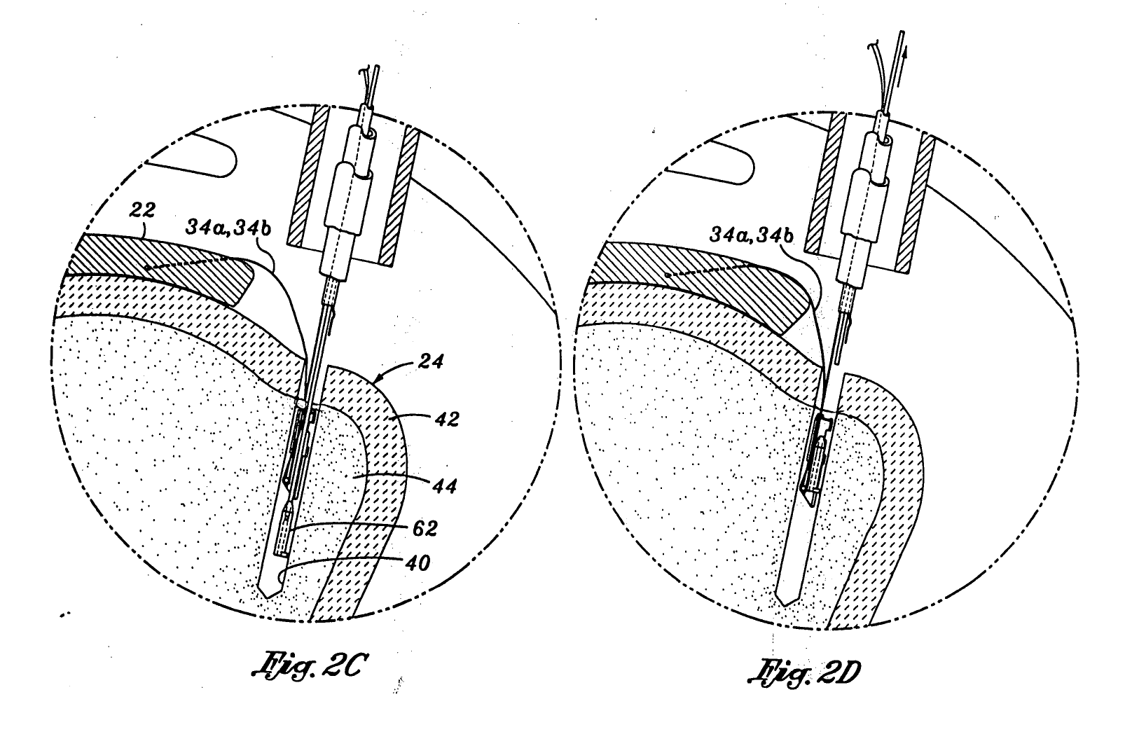

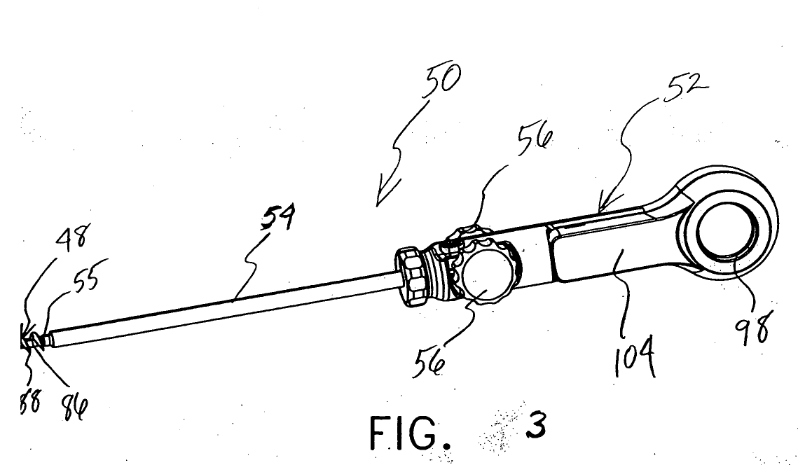

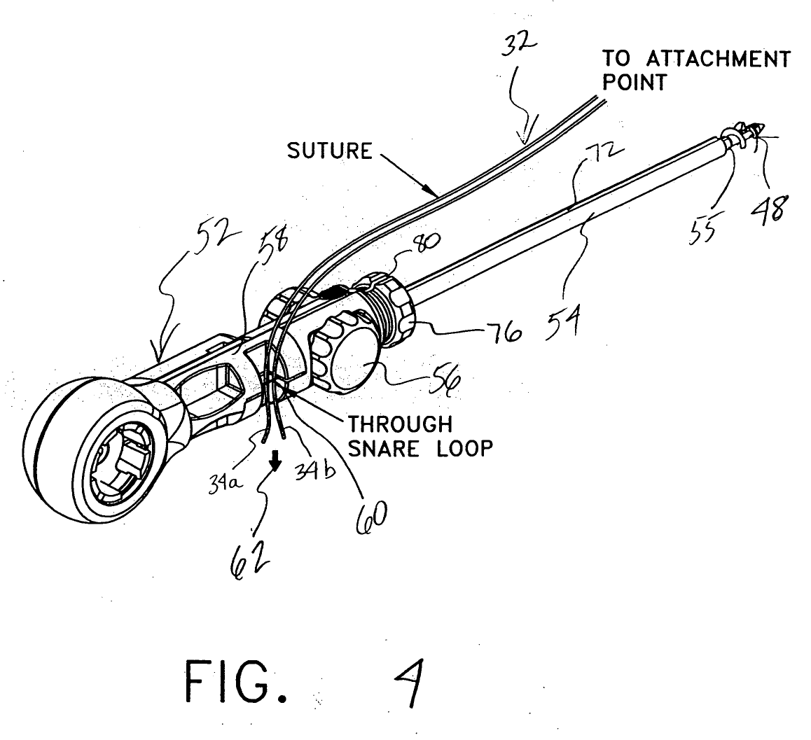

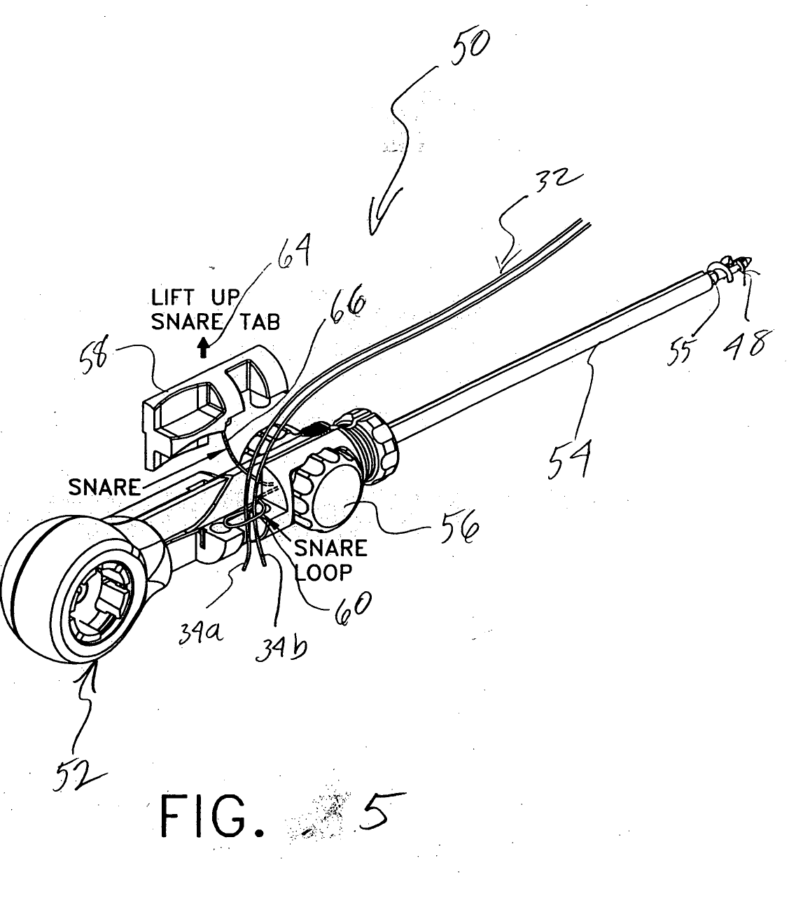

FIGS. 1A-1B and 2A-2D are cross-sectional views through the left shoulder of a human as viewed from the front and illustrate the use of an exemplary suture anchor system 20 for repairing a rotator cuff tendon injury. The rotator cuff tendon 22 is shown in its natural position overlying the bulbous humeral head 24 of the humerus bone 26. In rotator cuff injuries, the tendon 22 partially or completely separates from its attachment point to the humeral head 24, which point of attachment is typically located along an angled shelf, the greater tuberosity 28. In minimally invasive surgeries to repair the rotator cuff injury, the surgeon threads one or more sutures through the rotator cuff tendon 22 and anchors them to the greater tuberosity 28. The suture anchor system 20 of the present invention facilitates this latter step of anchoring the sutures to the greater tuberosity 28.

With reference first to FIG. 1A, a generally tubular trocar 30 provides a conduit through the soft tissue of the shoulder for the suture anchor system 20 of the present invention. Typically, the surgeon makes an incision or stab wound through the outer dermal layers of sufficient size to permit passage of the trocar 30 through skin and the deltoid muscle into proximity with the humeral head 24. Various trocars and techniques for creating the approach passageway are known and may be utilized with the present invention. In addition, more than one incision and conduit may be necessary to perform the several suturing and anchoring steps.

After establishing one or more direct conduits to the humeral head 24, the surgeon passes a length of suture through the soft tissue of the rotator cuff tendon 22 so that a loop 32 of suture material is embedded therein, as seen in FIG. 1B. The two free ends 34a, 34b of the length of suture are withdrawn from the patient and coupled to the suture anchor system 20. The specifics of this coupling and subsequent manipulation of the two free ends of the suture will be described more fully below. For the purpose of explaining the exemplary method of use, it is sufficient to understand that the two free ends 34a, 34b pass into a lumen at the distal end of the suture anchor system 20 and extend through the lumen in a proximal direction to a proximal end of the system to enable fixation or pulling of the suture ends. As seen in FIG. 1B, the two free ends 34a, 34b are shown projecting from a proximal end of the system.

The exemplary system 20 as illustrated is particularly suitable for anchoring a suture to a body cavity, specifically the humeral head 24 as shown. When anchoring sutures to such a bone structure, a conventional technique is to first form a blind hole or cavity 40 through the cortical layer 42 and into the soft cancellous matter 44, as seen in FIGS. 1A-1B and 2A-2D. The surgeon then inserts a suture anchor 46 into the cavity 40 and screws it in such that it cannot be removed from the cavity.

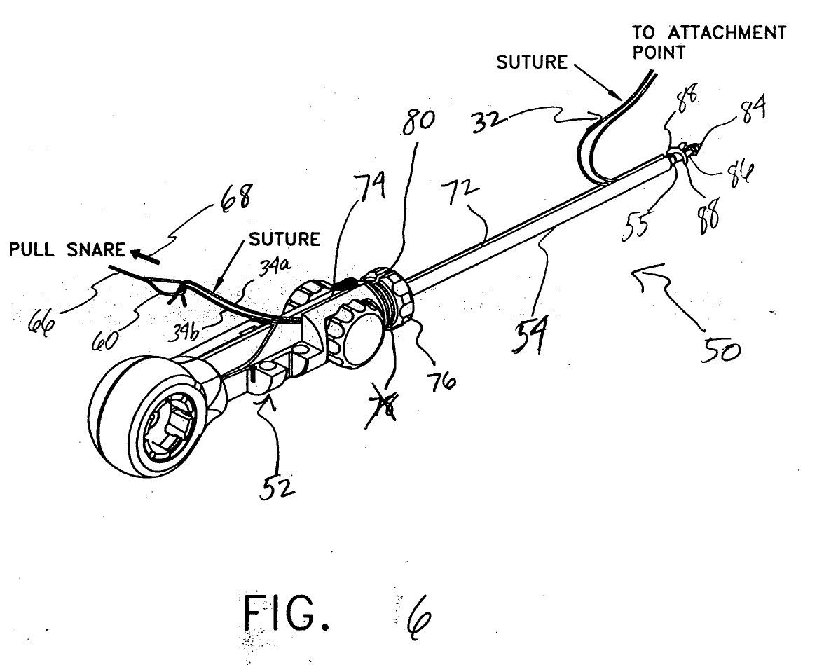

The suture anchor 46 performs two functions: anchoring itself within the body cavity and anchoring the sutures therein. In the disclosed embodiment, the former function is accomplished using a screw-type anchoring structure 48 (FIG. 3) located on the distal end of the suture anchor 46. The anchoring structure 48 will be described in more detail hereinbelow, but briefly, it functions to retain the suture anchor 46 within the cavity 40. In this manner, the suture anchor 46 is prevented from being removed from the cavity 40 by the anchoring structure 48. The present invention illustrates a particular anchoring structure 48, although any similar expedient will work.

The second function of the suture anchor 46 is the anchoring or fixation of the suture with respect to the suture anchor itself, without the use of knots. Desirably, the particular manner of anchoring the suture with respect to the suture anchor 46 permits easy adjustment of the length of suture between the suture anchor and the loop 32 formed in the soft tissue. This adjustment allows the surgeon to establish the proper tension in the length of suture for effective repair of the soft tissue; reattachment of the rotator cuff tendon 22 in the illustrated embodiment. In this regard, FIG. 2D shows the fully deployed suture anchor 46 after the free ends 34a, 34b have been placed in tension and locked within the suture anchor. Although not shown, the remaining steps in the procedure involve withdrawing the tube from the surgical site and severing the free ends 34a, 34b close to the suture anchor 46.

Now, with reference especially to FIGS. 3-12, a first embodiment of the present invention will be described. In FIG. 3 there is shown a suture anchoring device 50 which comprises a handle actuator 52 attached to an outer tubular shaft 54. An inner tubular shaft 55 is disposed within the outer tubular shaft 54. The screw-type anchor 48 is attached to a distal end of the inner shaft 55. The handle actuator 52 is adapted for both inserting the bone anchor 48 and for tensioning the suture 34, using suture cinching knob 56, in a manner to be discussed below.

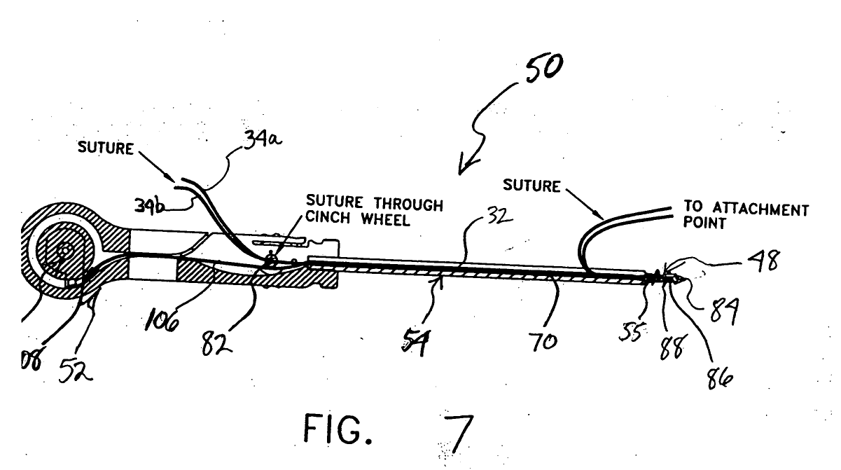

Now with reference in particular to FIG. 4, the handle actuator 52 is illustrated. The device 50 is located as desired within the blind hole or cavity 40 in the bone 26 (FIGS. 1-2). Then, a screw-type anchoring structure 48 is turned to become fixedly engaged with the bone 26. The screw-type anchoring structure 48 comprises a distal pointed end 84 and a proximal shaft 86, on the surface of which are a plurality of threads 88, such that the structure 48 resembles a conventional screw. To fixedly engage the structure 48 and the bone 26, therefore, the practitioner locates the anchoring structure 48 so that the distal end 84 is directly adjacent to the desired bone anchoring location. The handle 52 is then rotated in a clockwise direction, causing the attached shafts 54, 55 and screw shaft 86 to follow, so that the anchor 46 is advanced into the bone as the threads 88 are engaged therein. The mechanism is similar to that by which a screwdriver is rotated to engage a wood screw into a piece of wood. In the course of turning the anchor 46, a slot 72, which serves to allow the suture 32 to exit the interior of the shaft 54, is aligned so that the slot 72 is facing the tissue to be repaired.

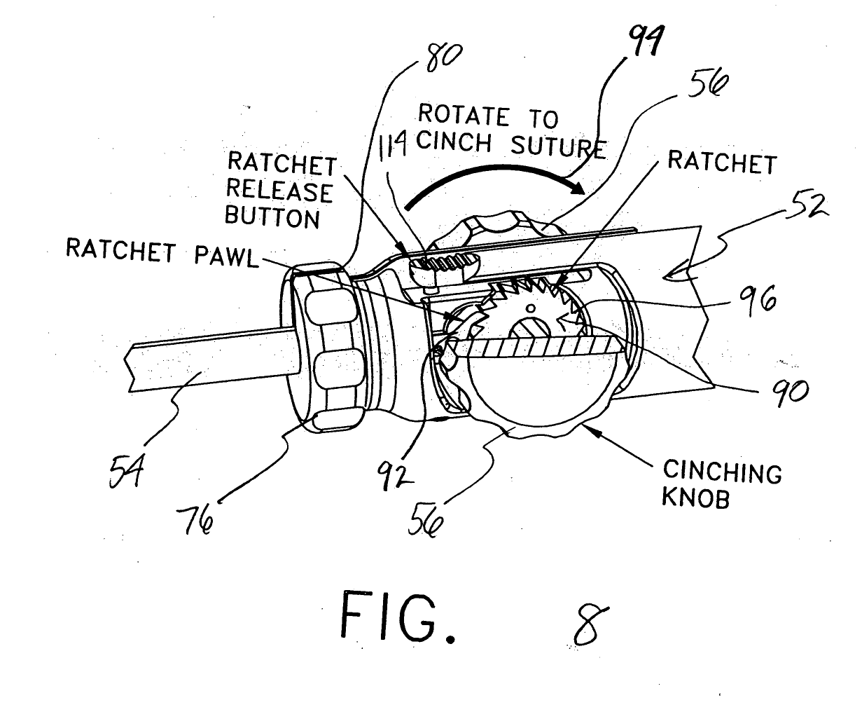

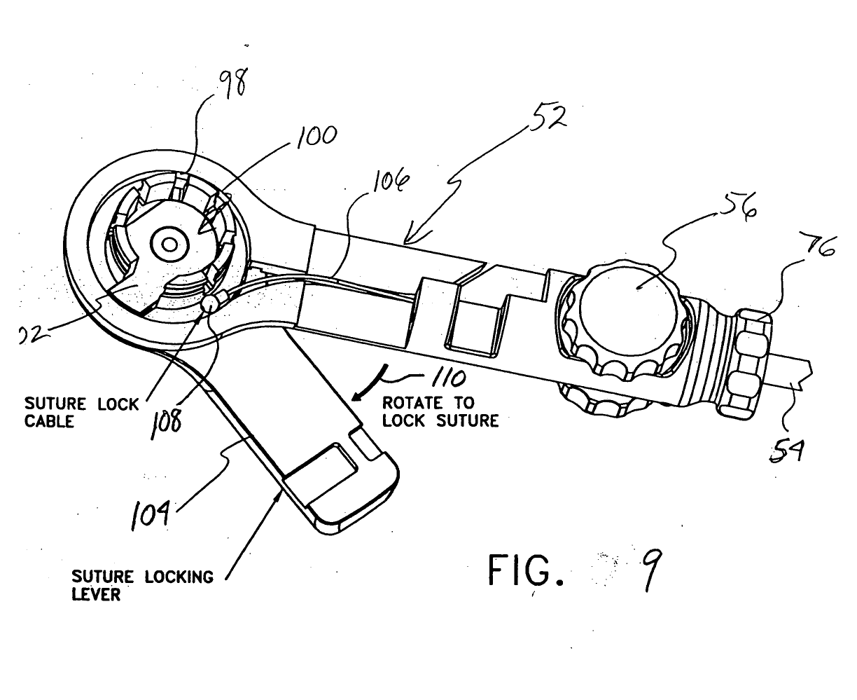

The handle actuator 52 comprises a snare tab 58, to which is secured a snare loop 60, which is actually more visible in FIG. 5. The next step in the procedure, once the suture loop 32 has been attached to the soft tissue 22, is to thread the suture 32 through the device 50. To accomplish this, the free ends 34a, 34b of the suture loop 32, which is attached to the soft tissue 22 (not shown in FIG. 4), are threaded through the snare tab 58 and attached snare loop 60 in a downward direction illustrated by the arrow 62. Then, as shown in FIG. 5, the snare tab 58 is lifted upwardly in a direction illustrated by arrow 64 to also lift upwardly an attached snare 66, which is connected to the snare tab 58. As shown in FIG. 6, the snare 66 and snare loop 60 are pulled in a generally upward and proximal direction illustrated by arrow 68, thus pulling the suture free ends 34a, 34b into the lumen 70 of the tubular shaft 55 and down into the suture anchor 46. Further, by pulling on the snare 66, the suture ends 34 are pulled back out of the suture anchor.

As noted by viewing FIGS. 5-7, the suture 32 is disposed within a lumen 70 (FIG. 7) in the inner tubular shaft 55. A slot 72 in the outer shaft 54, and a like slot in the inner shaft 55 (not shown), allow the suture 32 to be threaded into the suture anchor 46. The suture then extends proximally through the handle actuator 52, as shown, within an upper slot 74, which is continuous with the slot 72, as shown in FIG. 5. A suture anchor insertion knob 76 is disposed on the handle actuator 52, and includes a slot 80, which is arranged to align with the slots 72 and 74. Thus, the continuous slots 72, 80, and 74 permit ready insertion of the suture 32 into the lumen of the tube 55 and the interior of the handle actuator 52, as shown particularly in FIG. 7.

In FIG. 7, which is a cross-sectional view illustrating the path of the suture 32 through the device 50, it can be seen that the suture cinching knob 56 (FIG. 6) is operatively connected to a suture cinch wheel 82, through which the suture 32 is disposed. The suture cinching function will be described in more detail hereinbelow.

Now, with the suture anchoring device fixedly anchored within the bone cavity 40 by means of the above described engagement of bone anchor 46 and adjacent bone 26, and further with the suture attached to the soft tendon 22, and threaded through the suture anchoring device 50, the practitioner is free to tension the suture 32 as desired in order to approximate the tendon 22 to the adjacent bone.

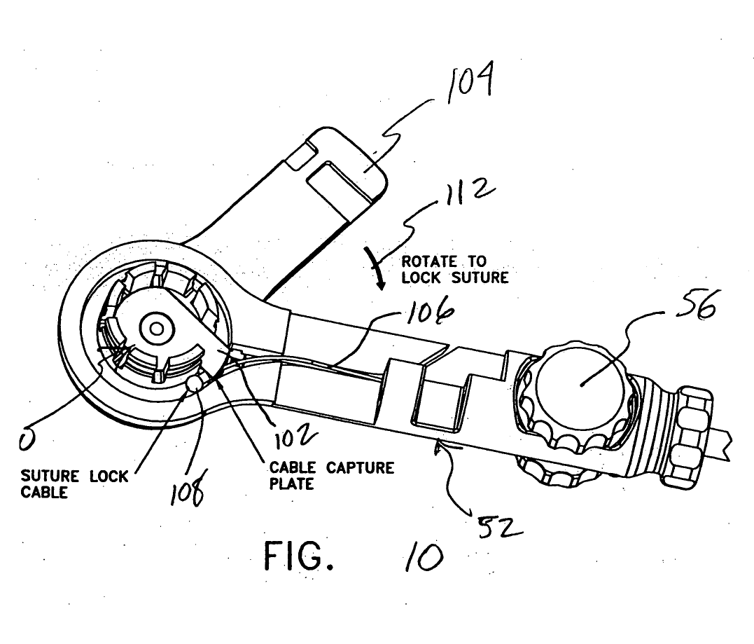

FIG. 8 illustrates the suture cinching mechanism which forms a part of the handle 52. As discussed above, opposed suture cinching knobs 56 operate together to actuate the cinch wheel 82 (FIG. 7) and an associated ratchet 90 and ratchet pawl 92. To cinch the suture 32 about the cinch wheel 82, the cinching knobs 56 are rotated in a clockwise direction, as illustrated by the arrow 94. As the ratchet 90 is rotated in concert with the cinching knobs 56, thus cinching the suture in order to tension it and therefore approximate the tendon 22 to the bone 26, the pawl 92 sequentially engages each tooth 96 on the ratchet 90 to prevent the ratchet from reversing. When the suture is cinched to a desired level, the pawl 92, through its engagement with a then corresponding tooth 96, will maintain the suture cinching mechanism in the desired position, to maintain the suture tension.

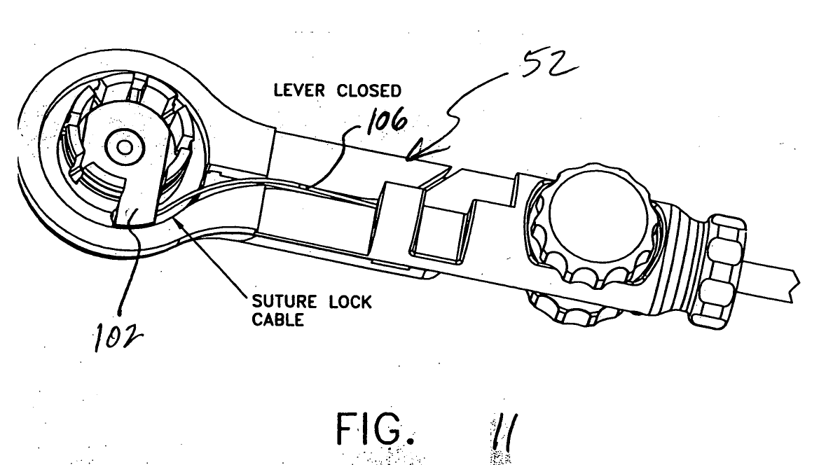

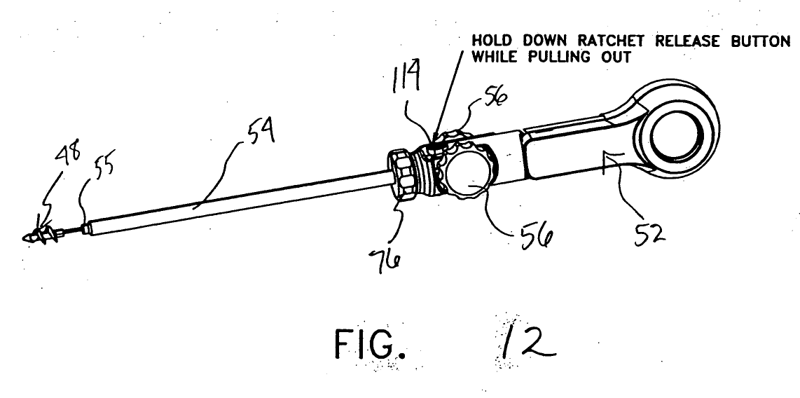

Now with particular reference to FIGS. 7 and 9-11, there are illustrated, in sequence, structure and steps for locking the suture in place once it has been tensioned to a desired level, as described above. The handle 52 comprises at its proximal end a recess 98 which extends entirely therethrough and is open on both sides. As shown particularly in FIG. 9, a suture locking mechanism 100 is disposed within the recess 98. The suture locking mechanism 100 comprises a rotatable cable capture plate 102, which rotates responsive to the actuation of a rotatable suture locking lever 104. A suture lock cable 106 extends into the recess 98, as shown, and has a stop member 108 disposed on its proximal end. The suture lock cable 106 extends distally through the handle 52 and the lumen 70 of the tubular shaft 55, as shown in FIG. 7. It is attached at its distal end to the bone anchor 48. To lock the suture 32 in place, the suture locking lever 104, which is normally stowed in a closed position against the handle 52, as shown, for example, in FIG. 3, is released and rotated in the direction indicated by an arrow 110 (FIG. 9). Rotation of the suture locking lever 104 results in a corresponding rotation of the cable capture plate 102. As shown in FIG. 10, the lever 104 continues to be rotated about the handle 52, in the direction of arrow 112, so that the cable capture plate 102 rotates as well, to the position shown, wherein it is distal to the stop member 108. When the lever 104 is fully circumferentially rotated, once again to its closed position against the handle 52, the cable capture plate 102 is engaged with the stop member 108, as shown in FIG. 11, to lock the cable 106 in place.

The action of locking the cable 106 in place, as illustrated in FIG. 11, functions to place the cable in tension, wherein it tends to move toward the center of the lumen 70 (FIG. 7).

Once the suture is locked in place, the practitioner can remove the apparatus 50, including all but the inner shaft 55, by depressing a ratchet release button 114 (FIGS. 8 and 12), and withdrawing the handle 52 proximally.

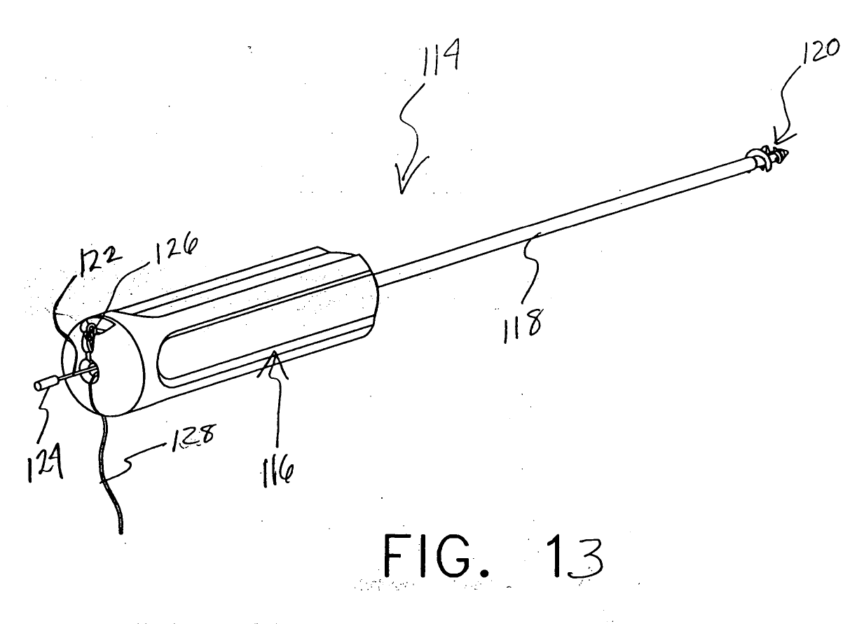

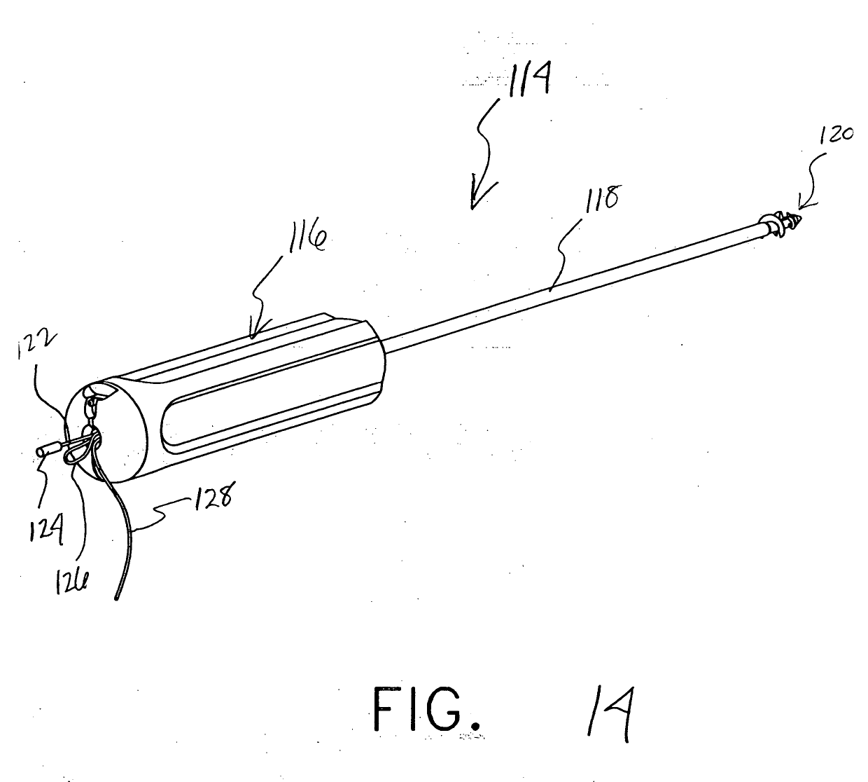

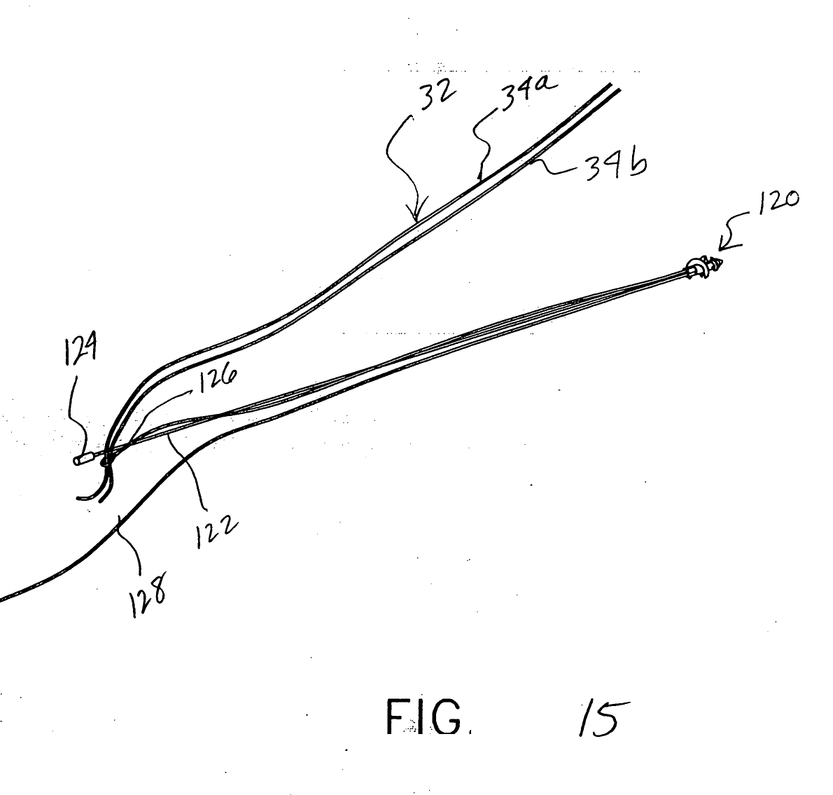

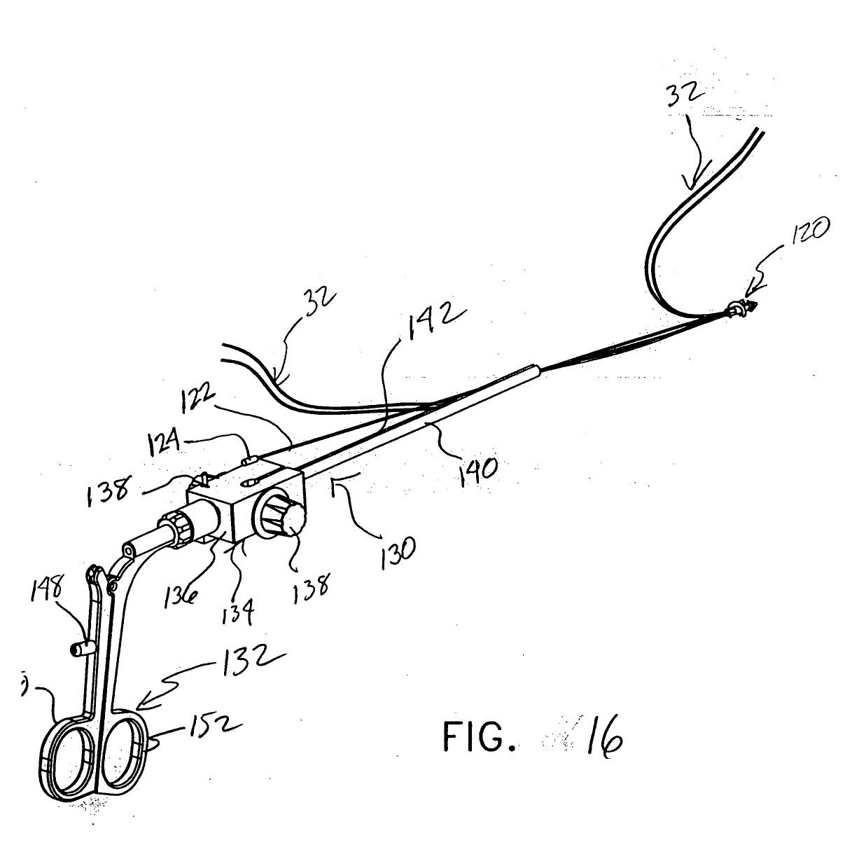

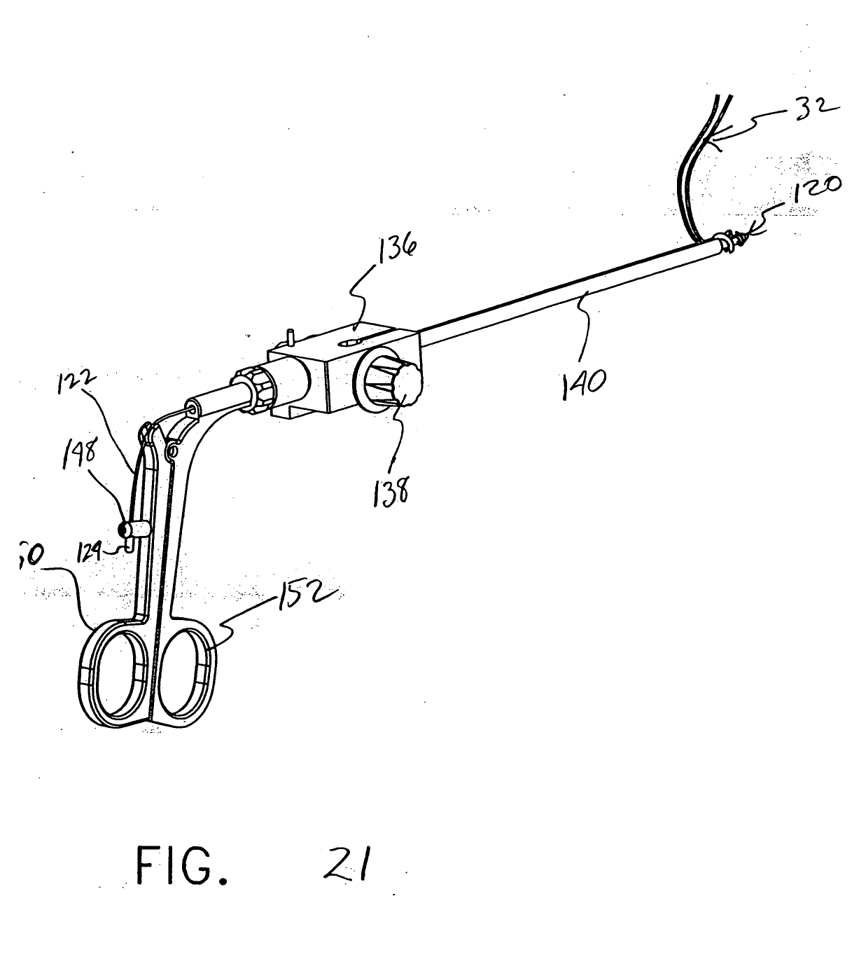

A second embodiment of the inventive apparatus is illustrated in FIGS. 13-22. In this embodiment, a primary difference is that separate bone anchor and suture tensioning devices are utilized. Thus, as shown in FIGS. 13 and 14, there is provided a bone anchor installation tool 114, which comprises a handle 116, a shaft 118 extending distally from the handle 116, and a screw-type bone anchor 120 disposed on a distal end of the shaft 118. A suture lock cable 122 having a stop member 124 disposed on a proximal end thereof extends from the proximal end of the handle 116. A snare loop 126 and a length of suture 128 are both attached to the bone anchor 120 and extend proximally through the shaft 118 and handle 116, as illustrated. As in the prior embodiment, the anchor 120 is located adjacent to a desired bone location, within the bone cavity 40 (FIGS. 1-2), and the handle 116 is rotated in a clockwise fashion to insert the bone anchor 120 into the adjacent bone. Then, the suture loop 32, previously attached to the soft tissue 22, is threaded through the shaft 118.

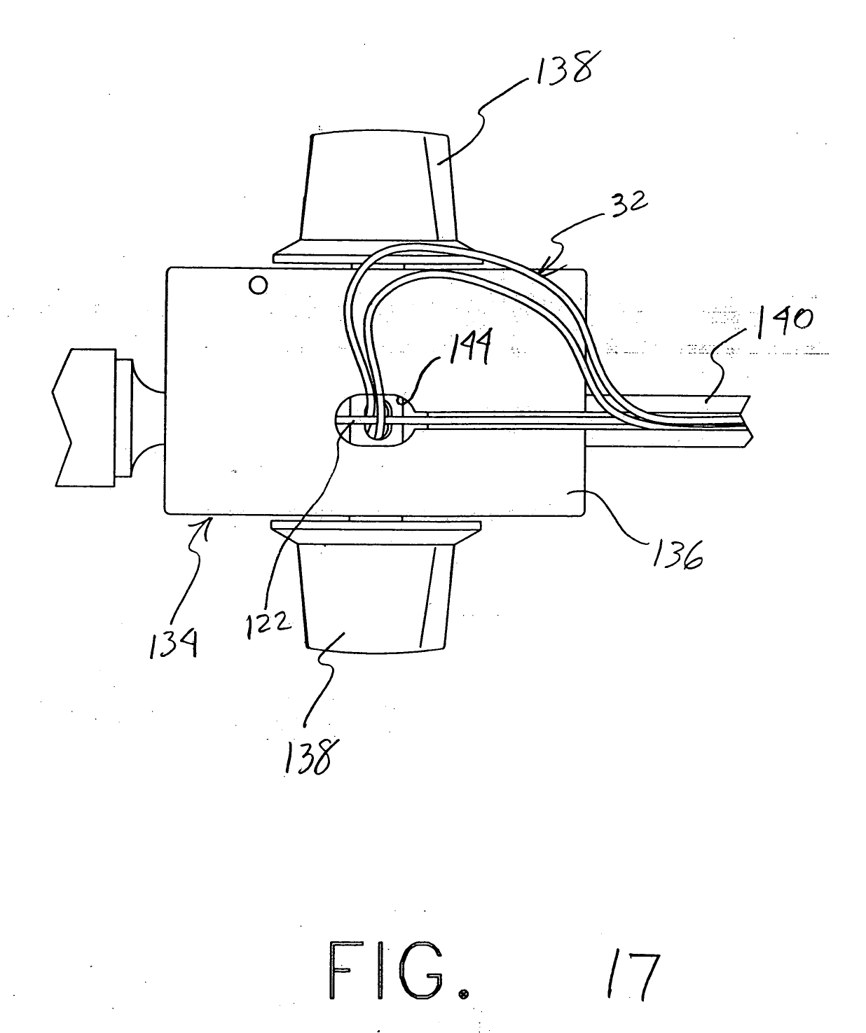

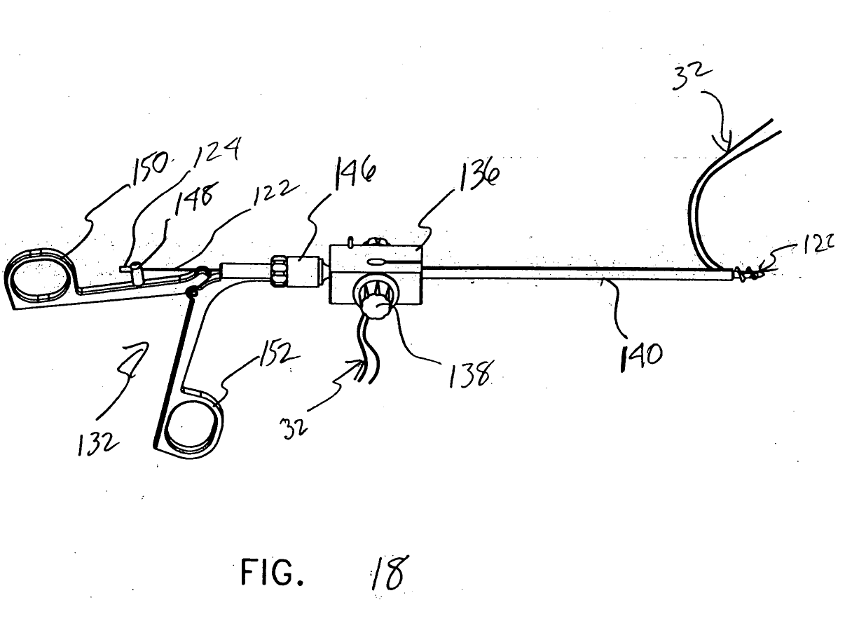

Once the bone anchor 120 is disposed in the desired bone, the device 114 is withdrawn from the procedural site, as shown in FIG. 15, leaving only the bone anchor 120 and attached suture 128, snare loop 126, and cable 122, as shown, wherein the suture loop 32 from the soft tissue 22 is disposed in the snare loop 126. The next step in the inventive procedure is to tension the suture loop 32, as desired, to approximate the soft tissue 22 to the bone 26. In order to accomplish the tensioning step, a suture tensioning apparatus 130 is inserted into the procedural site, and engaged with the suture 32, as shown in FIG. 16. The apparatus 130 comprises a handle portion 132, and a tensioning device 134, which comprises a housing 136 and suture cinching knobs 138. Distally of the suture tensioning housing 136 is a tubular shaft 140 having a slot 142 on an upper end thereof. The suture 32 and cable 122 are inserted into the tubular shaft through the slot 142, as shown. FIG. 17 shows the tensioning device 134 from the top, wherein the suture loop 32 has been inserted into the housing 136, through an accommodating aperture 144.

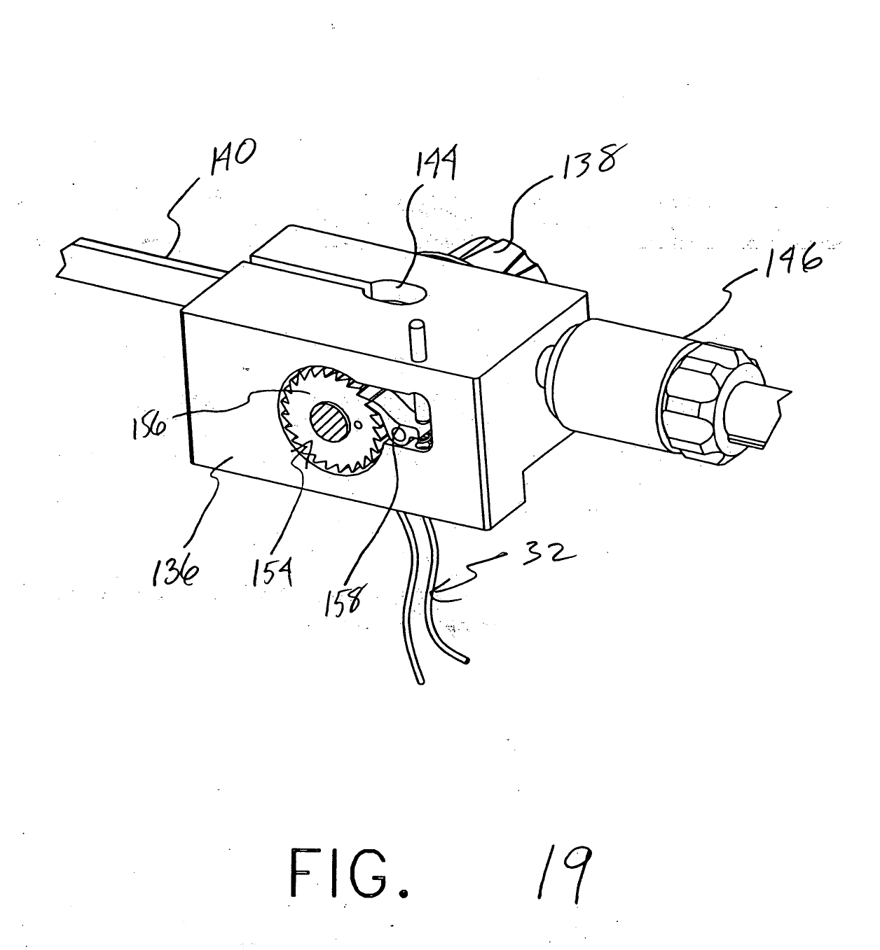

As shown in FIG. 18, the cable 122 has been extended proximally from a proximal end of the suture tensioning housing 136, through a cylinder 146, and then through a post 148 which is disposed on a first handle 150 of the handle portion 132. As can be seen by a comparison of FIGS. 16 and 18, the first handle 150 has been pivotably moved apart from a second handle 152 in FIG. 18, to permit the post 148 to be in alignment with the cable 122.

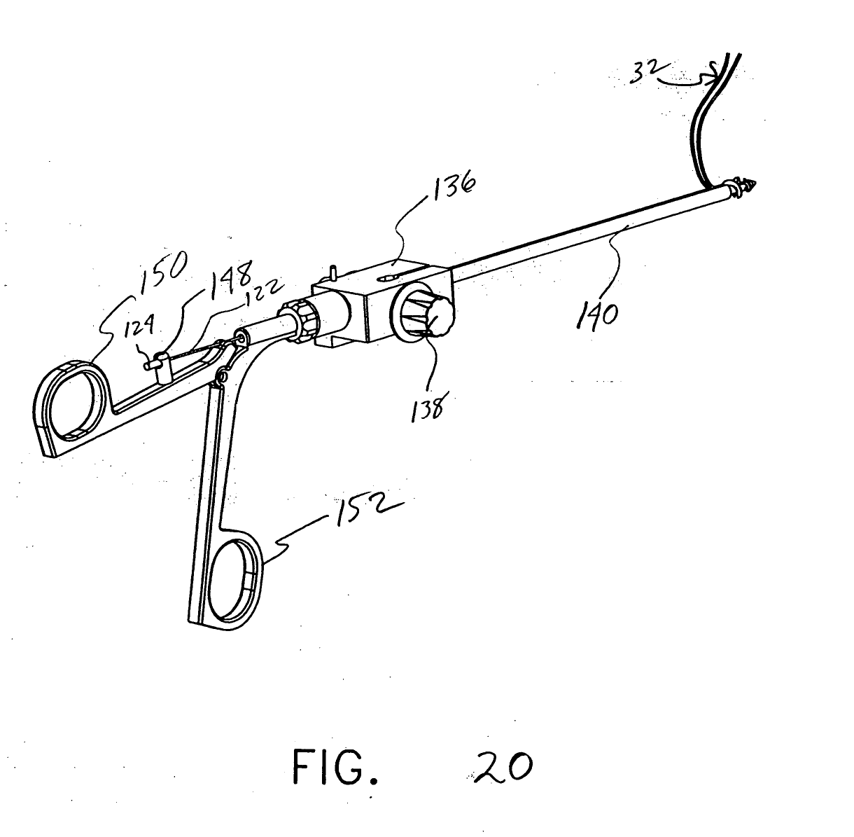

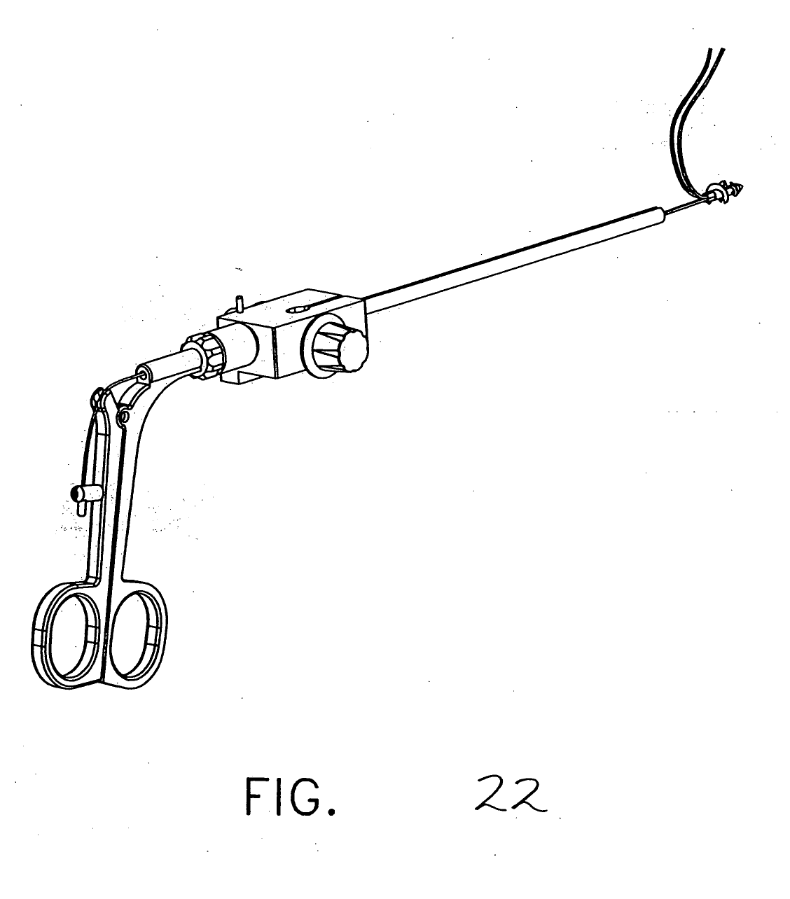

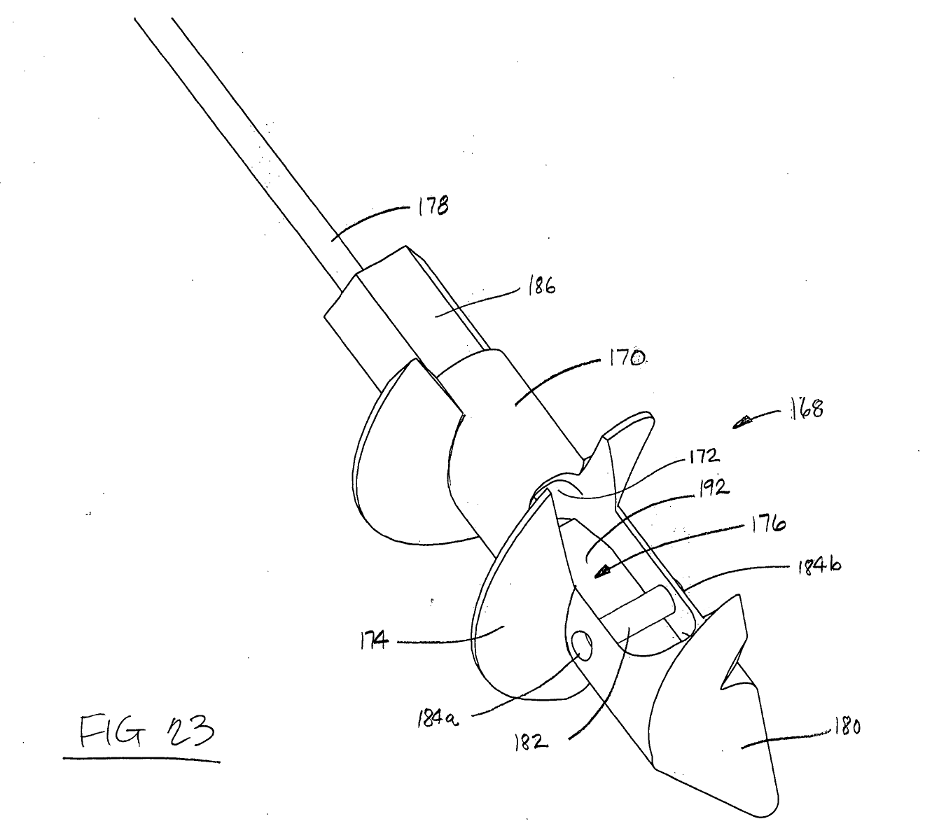

FIG. 19 illustrates the same housing 136 as is illustrated in FIG. 17, but one of the tensioning knobs. 138 has been removed in FIG. 19 for clarity, thereby revealing a suture tensioning mechanism 154 which is very similar to that employed in the embodiment of FIGS. 3-12, including, for example, a ratchet 156 and a ratchet pawl 158. As shown, the suture 32 has been threaded through the suture tensioning mechanism 154, about a suture cinch wheel (not shown), in much the same manner as in the first embodiment. Once the suture has been tensioned, as desired, using the suture cinching knobs 138, in the same manner as in the prior embodiment, then, as shown in FIG. 21, the handle 150 is pivoted downwardly to a location adjacent to the handle 152, thereby pulling the cable 122 proximally, and causing it to clamp the suture 32 within the shaft 140, between the cable 122 and lumen walls, as is the case with the prior embodiment. Then, as shown in FIG. 22, the device 130 may be withdrawn from the procedural site.

The figures above illustrate the delivery and actuation mechanisms associated with the installation and deployment of a specially designed implant for fixation of soft tissues to bone. This unique implant has been adapted to a screw body for excellent holding power in soft cancelleous bone. Although there are many features of this implant that are similar to that disclosed in U.S. patent application Ser. No. 09/781,793, already incorporated by reference in the present application, there are new features specific to the suture locking mechanism that will be described by referring to the figures below.

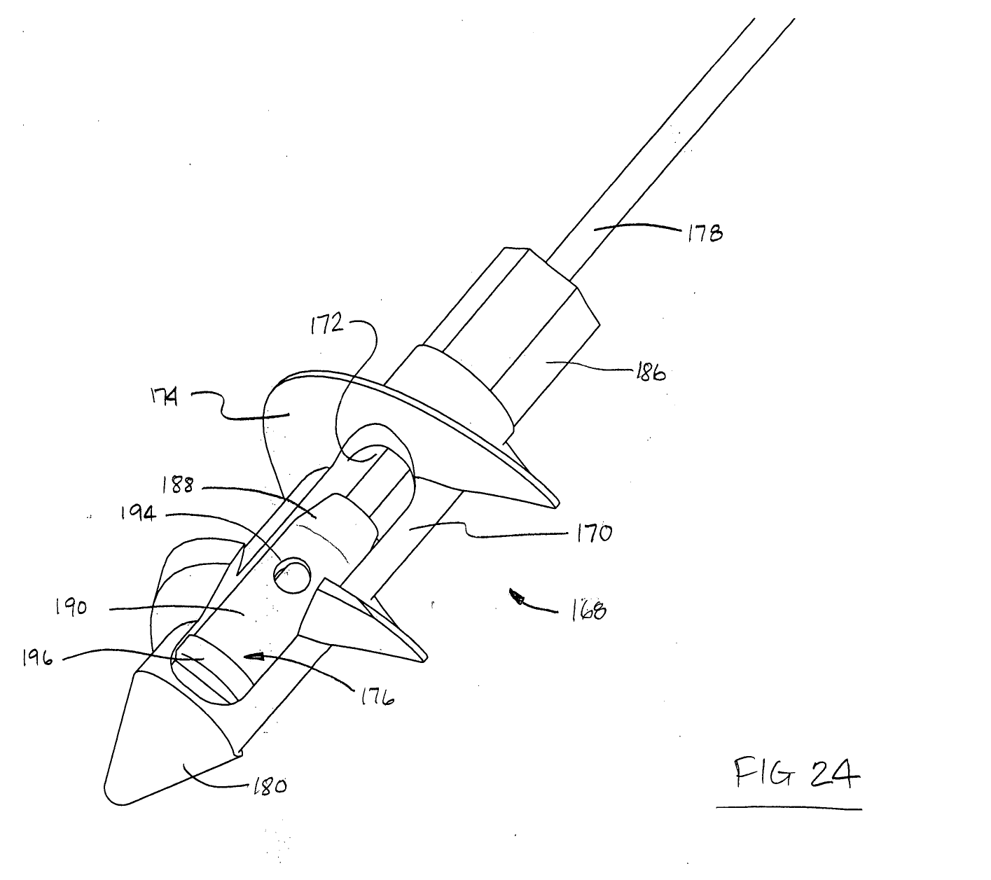

Referring now to FIGS. 23 and 24, there may be seen a knotless suture anchor 168 similar in structure to suture anchor 46 in FIG. 1B, comprising an anchor body 170, a lumen 172 through anchor body 170, screw threads 174, suture locking plug 176, and suture lock cable 178. The suture anchor 170 further comprises a nose 180, pulley 182, which is disposed in holes 184a, b, and a hex drive 186. As previously described, the hex drive 186 is used to screw the suture anchor 168 into, for instance, bone for the purpose of creating a suture attachment point. The screw threads 174 accomplish the task of retaining the suture anchor 168 in the cancelleaous portion of the bone, although the threads may also bear upon the underside of the cortical surface.

The suture locking plug 176 includes a tapered nose 188, a body 190, a tapered locking surface 192, a weld hole 194, and a travel stop 196. A suture lock cable 178 is inserted into the locking plug 176 such that the distal end of the suture lock cable 178 is visible through the weld hole 194. These two structures, the suture lock cable 178 and locking plug 176 may be joined together via a weld in the weld hole 194 by laser welding or other suitable means. The mechanism for joining these two structures is not critical, as they may be joined in any manner sufficient to allow the plug 176 to be pulled by the cable 178 with a force sufficient to lock sutures, and to allow the two structures to disassociate from each other to allow the deployment system to be withdrawn from the operative site. Such methods may include welding adhesive bonding, insert molding, overmolding, and similar known approaches.

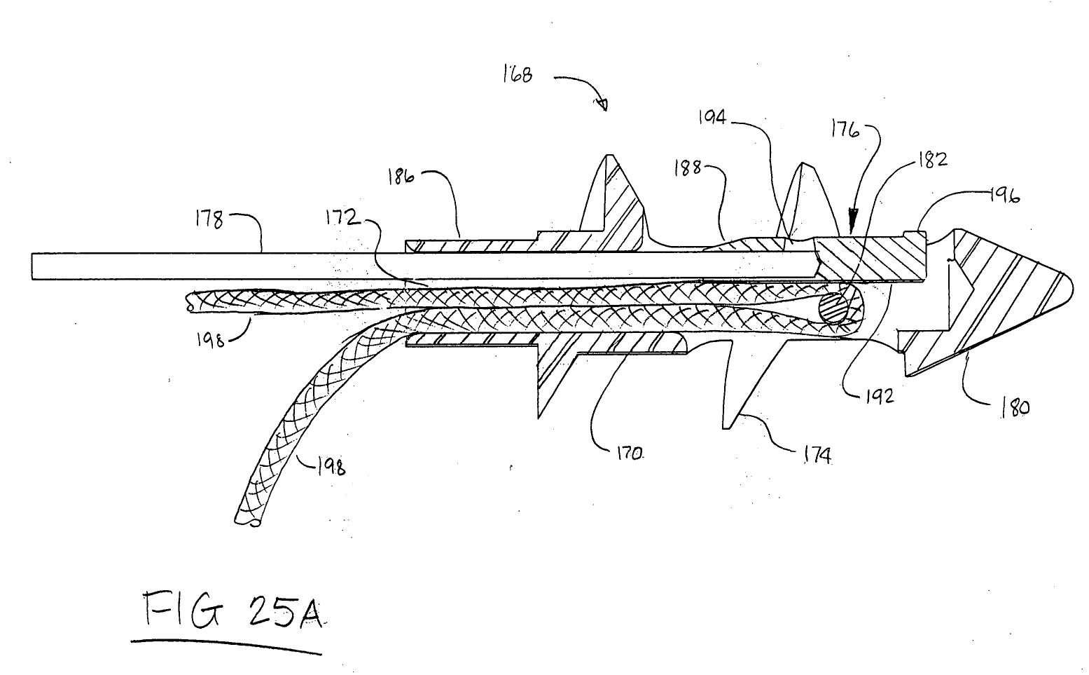

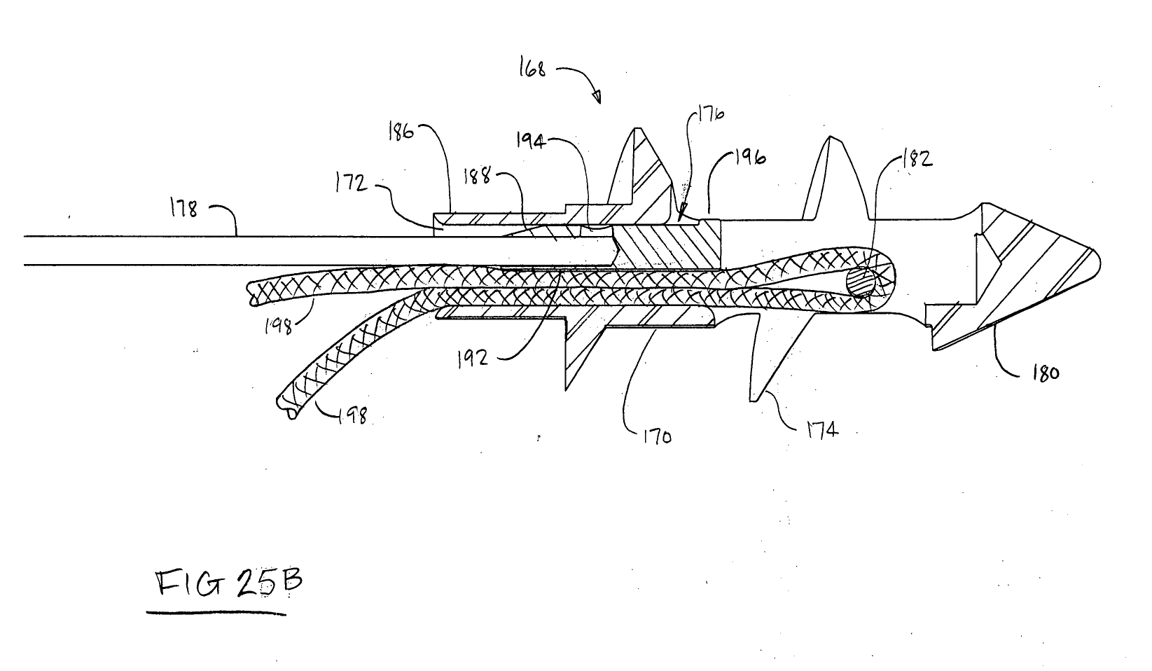

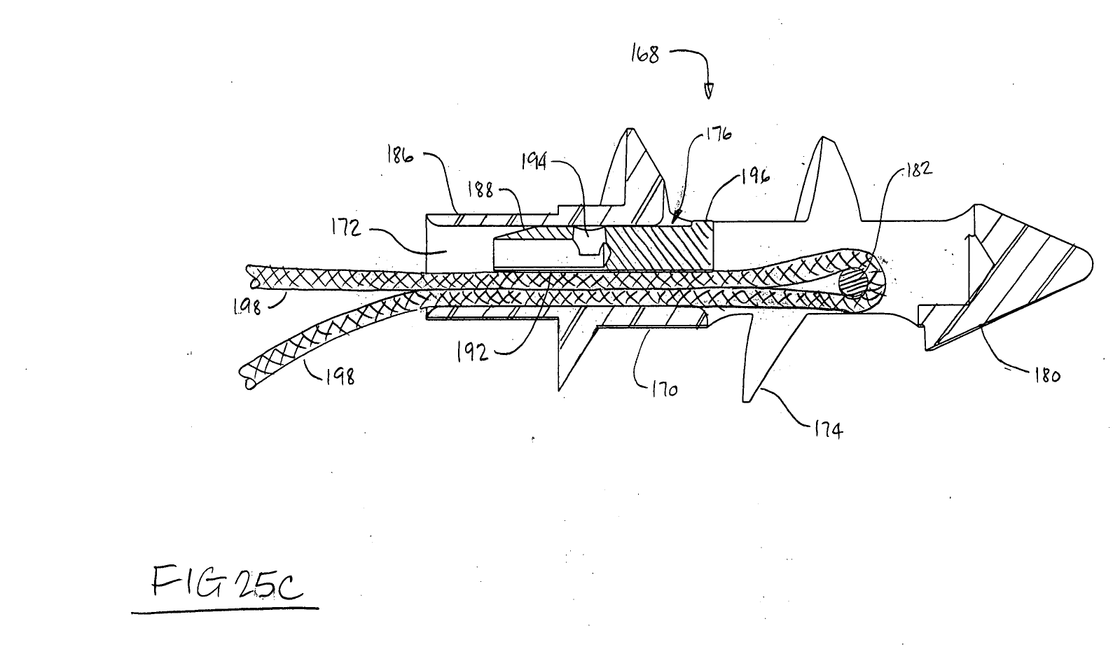

Referring now to FIGS. 25A, 25B, and 25C, there is seen a sequence of cross-sectional views of the suture anchor 168, illustrating the suture locking function of the present invention. These figures include a suture strand 198 that is disposed in the lumen 172 and around the pulley 182. It is to be understood that the suture strand 198 is representative of one or more suture strands that may be threaded into the suture anchor 168, and that the structure is not limited to accepting just a single strand. In fact, in the exemplary embodiment, two strands of suture are disposed within the suture anchor 168. However, a single strand is illustrated herein for clarity.

Now with reference particularly to FIG. 25A, there is clearance between the walls of the lumen 172 and the suture strand 198 that allow the suture strand 198 to move freely within the lumen 172 and around the pulley 182. In this configuration, the suture may be tensioned as previously described to approximate the soft tissues to be repaired to the bone or other tissues. By pulling on the suture lock cable 178, the locking plug 176 is forced to follow into the lumen 172. The tapered nose 188 facilitates leading the suture locking plug 176 into the lumen 172 as the suture lock cable is pulled. It may be seen in FIG. 25B that the tapered locking surface 192 is in intimate contact with the suture strand 198, and fills the lumen 172 such that a frictional lock between the lumen 172, the plug 176, and the suture 198 is created. The tapered locking surface 192 is tapered in order to accommodate dimensional tolerances in the diameter of the suture 198 and the lumen 172.

As may be seen by referring to FIG. 25C, the suture lock cable is no longer attached to the plug 176. This is a result of the frictional force created between the lumen 172, the plug 176, and the suture 198 overcoming the tensile strength of the attachment, for example the weld described above, between the suture lock cable 178 and the suture plug 176. When the cable 178 and plug 176 are disassociated, the deployment means described previously may be removed from the operative site, leaving the knotless suture anchor 168 and suture 198 in place, securing the tissues. The travel stop 196 is disposed on the plug 176 to prevent the plug 176 from being pulled completely through the lumen 172 in the event that the frictional lock does not generate sufficient force to break the attachment of the suture lock cable 178 to the suture plug 176.

Accordingly, although an exemplary embodiment of the invention has been shown and described, it is to be understood that all the terms used herein are descriptive rather than limiting, and that many changes, modifications, and substitutions may be made by one having ordinary skill in the art without departing from the spirit and scope of the invention. In particular, it is noted that the procedures, while oriented toward the arthroscopic repair of the rotator cuff, are applicable to the repair of any body location wherein it is desired to attach or reattach soft tissue to bone, particularly using an arthroscopic procedure.

What is claimed is:

1. A knotless suture anchor apparatus for anchoring a length of suture with respect to a body cavity, comprising:
an anchor body having an anchoring structure for fixing the anchor body within a body cavity, the anchoring structure comprising a threaded surface which is rotatable to engage adjacent bone;
a suture tensioning mechanism for accommodating and tensioning said length of suture; and
a suture locking mechanism for locking said length of suture in place once it has been tensioned to a desired level.

2. The apparatus as recited in claim 1, wherein said threaded surface is disposed on a distal end of said anchoring structure, said anchoring structure further comprising a shaft extending proximally from said threaded surface.

3. The apparatus as recited in claim 2, wherein a proximal end of said shaft is connected to a handle.

4. The apparatus as recited in claim 3, wherein said shaft comprises an inner, tubular shaft and said anchoring structure further comprises an outer shaft disposed about said inner shaft.

5. The apparatus as recited in claim 4, wherein said outer shaft is proximally removable from its position disposed about said inner shaft after the threaded surface is engaged in the adjacent bone.

6. The apparatus as recited in claim 1, wherein said suture tensioning mechanism is structurally integrated with said anchoring structure.

7. The apparatus as recited in claim 1, wherein said suture tensioning mechanism is deployed after the threaded surface is engaged in the adjacent bone and after portions of said anchoring structure have been withdrawn.

8. The apparatus as recited in claim 1, and further comprising a snare loop for snaring said length of suture and threading it through said suture tensioning mechanism.

9. The apparatus as recited in claim 1, wherein said suture tensioning mechanism comprises a rotatable knob which is operably connected to a ratchet and pawl system.

10. The apparatus as recited in claim 1, wherein said suture locking mechanism comprises a locking lever for actuating a rotatable cable capture plate.

11. A knotless suture anchor apparatus for anchoring a length of suture with respect to a body cavity, comprising:
an anchor body having a screw-type anchoring structure for fixing the anchor body within a body cavity, the screw-type anchoring structure comprising a threaded surface on a distal end thereof which is rotatable to engage adjacent bone, said anchoring structure further comprising a shaft extending proximally from said threaded surface; and
a handle connected to a proximal end of said shaft.

12. The apparatus as recited in claim 11, and further comprising a suture tensioning mechanism for accommodating and tensioning said length of suture.

13. The apparatus as recited in claim 12, and further comprising a suture locking mechanism for locking said length of suture in place once it has been tensioned to a desired level.

14. A method of securing soft tissue with respect to a body cavity without knots, comprising:
passing a length of suture through soft tissue so that a loop of suture material is embedded in the soft tissue resulting in two free ends;
engaging a distal end of the anchor body with adjacent bone to fix the anchor body in place within the body cavity;
threading the two free ends of the length of suture through an anchor body;
tensioning the length of suture to approximate the soft tissue to the bone as desired; and
locking the length of suture in position after it has been tensioned as desired.

15. The method as recited in claim 14, wherein said threading step includes snaring said length of suture.

16. The method as recited in claim 14, and further comprising removing a portion of said anchor body after said engaging step.

17. The apparatus as recited in claim 4, wherein the inner shaft is receivable within and detachably connected to said outer shaft.

18. The apparatus as recited in claim 17, wherein the outer shaft includes a polygon-shaped cavity to detachably connect with inner shaft.
